SEQUENCE LISTING (3 sequences)

SEQ ID NO: 1 - DNA, 19 bp, synthetic construct
  misc_feature 1..19: note = ITS1
  source 1..19: mol_type = other DNA
tccgtaggtgaacctgcgg

SEQ ID NO: 2 - DNA, 20 bp, synthetic construct
  misc_feature 1..20: note = ITS4
  source 1..20: mol_type = other DNA
tcctccgcttattgatatgc

SEQ ID NO: 3 - DNA, 1129 bp, Dunaliella salina
  misc_feature 1..1129: note = Dunaliella salina OH214
  source 1..1129: mol_type = genomic DNA
atgcatgtctaagtattaaacttgctttatactgtgaaactgcgaatggcttcattaaatcagttatagtttatttgatggtacctttactcggataaccgtagtaattctagagctaatacgtgcgtaaatcccgacttctggaagggacgtatttattagataaaaggccagccgggcttgcccgactcttggcgaatcatgataacttcacgaatcgcacggctttgtgccggcgatgtttcattcaaatttctgccctatcaactttcgatggtaggatagaggcctaccatggtggtaacgggtgacggaggattagggttcgattccggagagggagcctgagaaacggctaccacatccaaggagggcagcaggcgcgcaaattacccaatcccaacacggggaggtagtgacaataaataacaataccgggcatttttgtctggtaattggaatgagtacaatctaaatcccttaacgagtatccattggagggcaagtctggtgccagcagccgcggtaattccagctccaatagcgtatatttaagttgttgcagttaaaaagctcgtagttggatttcgggtgggttgtagcggtcagcctttggttagtactgctacggcctacctttctgccggggacgagctcctgggcttaactgtccgggactcggaatcggcgaggttactttgagtaaattagagtgttcaaagcaagcctacgctctgaatacattagcatggaataacacgataggactctggcttatcttgttggtctgtaagaccggagtaatgattaagagggacagtcgggggcattcgtatttcattgtcagaggtgaaattcttggatttatgaaagacgaacttctgcgaaagcatttgccaaggatgttttcattaatcaagaacgaaagttgggggctcgaagacgattagataccgtcgtagtctcaaccataaacgatgccgactagggattggcaggtgtttcgttgatgaccctgccagcaccttatgagaaatcaaagttttgggttccggggggagtatggtcgcaaggctgaaacttaaggaatgagggaagggcaccacagcggtttaacctagcggcagct

(19) United States
(12) Patent Application Publication
(10) Pub. No.: US 2024/0101954 A1
(43) Pub. Date: Mar. 28, 2024

(54) NOVEL DUNALIELLA SALINA AND USES THEREOF

(71) Applicant: IUCF-HYU (INDUSTRY-UNIVERSITY COOPERATION FOUNDATION HANYANG UNIVERSITY), Seoul (KR)

(72) Inventors: Eon Seon JIN, Seoul (KR); Hyeon Jun OH, Incheon (KR); Min Jae KIM, Seoul (KR)

(21) Appl. No.: 18/518,641

(22) Filed: Nov. 24, 2023

Related U.S. Application Data
(63) Continuation-in-part of application No. PCT/KR2022/007255, filed on May 20, 2022.

(30) Foreign Application Priority Data
May 25, 2021 (KR) 10-2021-0066938

(51) Int. Cl. C12N 1/12; A23K 10/16; A23L 17/60; C12P 23/00

(52) U.S. Cl. CPC C12N 1/125; A23K 10/16; A23L 17/60; C12P 23/00; C12R 2001/89

(57) ABSTRACT

The present invention relates to a Dunaliella salina OH214 strain having an improved ability to produce pigments, and in particular lutein. As it is possible to produce carotenoid pigments, specifically xanthophylls, with the Dunaliella salina OH214 strain by consuming less energy, it is possible to efficiently produce pigments at an industrial level. In addition, the Dunaliella salina OH214 strain can be applied as a raw material for foods, health functional foods, and pharmaceuticals, containing pigments. Moreover, the Dunaliella salina OH214 strain is a domestic native microalga in Korea and thus does not cause any GMO issues, it is possible to reduce costs by using seawater as a culture medium in Korea, and the effect of related industrial development can be expected.

### STATEMENT DESIGNATING GRACE PERIOD INVENTOR DISCLOSURES

Applicant informs that the subject matter of this utility was disclosed by the inventor or joint inventor or by another who obtained the subject matter disclosed directly or indirectly from the inventor or joint inventor one year or less than before the effective filing date of a claimed invention which do not qualify as prior art under 35 U.S.C. 102(b)(1) for the followings: (1) H. OH, et al., KSMB-Annual Meeting & Symposium, 2020 Yeosu Venetian Hotel, South Korea, Nov. 12, 2020 and (2) H. OH, Dunaliella salina OH214: A promising strain for lutein production, isolated from the Geumhong salt pan in the WestSea of Korea Thesis for the Master of Science, February 2021.

### INCORPORATION OF SEQUENCE LISTING

This application contains a sequence listing submitted in Computer Readable Form (CRF). The CRF file containing the sequence listing entitled “PK003086437.xml,” which was created on Dec. 12, 2023, and is 5,648 bytes in size. The information in the sequence listing is incorporated herein by reference in its entirety.

### TECHNICAL FIELD

The present invention relates to a Dunaliella salina strain having the ability to produce pigments and a use thereof, and specifically, relates to a new strain having the ability to produce pigments, a composition including the same, and a method for producing a food, a food raw material, or a pigment using the same.

### BACKGROUND ART

The retina is a light-sensitive tissue, and the structure thereof consists of a high proportion of unsaturated fatty acids, which may be easily damaged by reactive oxygen species. Lutein and zeaxanthin are xanthophyll-based pigments and found in the macular region of the human eye, and play a role in protecting the eye from damage induced by reactive oxygen species. In particular, lutein is found in a wider range than zeaxanthin (Non-Patent Document 1).

Xanthophyll-based pigments are known to play a role in blocking harmful rays such as ultraviolet rays and blue light and protecting the eyes from oxidative damage caused by reactive oxygen species, and thus are called macular pigments. Examples of a pigment belonging to xanthophylls include lutein, zeaxanthin, or the like. Lutein is known to act as an antioxidant that protects the inside of the eye from damage caused by oxygen free radicals that are naturally produced in the body. In particular, light with a wavelength of 400 to 500 nm damages the retina, and the maximum absorption wavelength of lutein is 445 to 472 nm Therefore, lutein may absorb blue light to reduce the intensity of light reaching the retina, thereby reducing oxidative damage (Non-Patent Document 2). Further, lutein kills cancer cells by reducing the growth of blood vessels that supply cancer tumors, and is also known to have some effect on the prevention of breast cancer, colon cancer, lung cancer, ovarian cancer, and skin cancer.

Therefore, maintaining the levels of lutein and zeaxanthin is critical to lowering the risk of macular degeneration. However, animals cannot produce xanthophyll and can obtain xanthophyll only through food intake, and these xanthophylls are present together with chlorophyll and carotene in the green parts of plants such as leaves, flowers, and fruits. Recently, a health functional food for eye health, including xanthophylls, and the like has attracted attention.

Existing marigold flowers are a representative source of zeaxanthin and lutein, and extraction from other higher plants has also been studied. In addition, zeaxanthin and lutein are also produced by genetically mutating the pigment synthesis mechanism in bacteria. Studies have also been conducted to obtain these pigments from microalgae. Among these conventional raw materials, marigold flowers have a disadvantage in that it takes a long time to breed flowering plants for production, and have a problem in that the production unit cost is high because the production amount is not large as compared to the land area for production.

In order to solve these problems, the development of zeaxanthin and lutein-producing algae into which a pigment synthesis mechanism was inserted using a bacterial system for replacing a higher plant system was achieved, but there is a problem in that a pigment obtained from bacteria is ultimately unsuitable for use as a food additive. In addition, since genetically modified organisms (GMOs) using gene insertion technology and the like are not preferred in the domestic market, this is a fatal disadvantage in the food additive market where consumer awareness is important, and as with higher plant systems, there is a problem in that the cost of maintaining a bacterial culture solution, a bioreactor, or the like may be high.

In the case of methods for obtaining these pigments from microalgae, it is difficult to use microalgae for optimal lutein production because microalgae in the related art have a limited ability to produce lutein, and in the case of genetically mutated microalgae, there are problems with using the microalgae as food or health functional food raw materials due to GMO issues when foreign genes, and the like are introduced.

Therefore, there is still a need for the development of microorganisms that do have restrictions for use as strains producing raw materials for foods, functional health foods, or pharmaceuticals.

### RELATED ART DOCUMENTS

### Non-Patent Documents

(Non-Patent Document 1) Handelman, Garry J, D Max Snodderly, Alice J Adler, Mark D Russett, and Edward A Dratz. 1992. ‘Measurement of carotenoids in human and monkey retinas,’ Methods in enzymology (Vol. 213, pp. 220-230).
(Non-Patent Document 2) Roberts, Richard L, Justin Green, and Brandon Lewis. 2009. ‘Lutein and zeaxanthin in eye and skin health,’ Clinics in Dermatology, 27: 195-201.

### DISCLOSURE

### Technical Problem

An object of the present invention is to provide a microorganism having a lutein-producing ability capable of providing xanthophyll pigments used as raw materials for foods, pharmaceuticals, and the like in the related art, a composition including the same, and a method for producing xanthophyll pigments using the same.

### Technical Solution

In order to achieve the object described above, the present inventors have made efforts to utilize strains that grow naturally in Korea as lutein-producing strains, rather than using genetic recombination methods that may cause problems in the food industry, and as a result, the present inventors isolated/identified a naturally occurring domestic strain that has an excellent ability to produce lutein, as well as the ability to produce zeaxanthin and β-carotene, compared to the genus Dunaliella in the related art, particularly, a Dunaliella salina strain, and confirmed a method for producing pigments using the same, thereby completing the present invention.

In this respect, the present invention provides Dunaliella salina OH214 (KCTC14434BP).

The Dunaliella salina OH214 has the ability to produce lutein.

The Dunaliella salina OH214 may further have the ability to produce zeaxanthin and/or β-carotene.

Further, the present invention provides a culture of Dunaliella salina OH214 (KCTC 14434BP).

In addition, the present invention provides a composition including Dunaliella salina OH214 (KCTC 14434BP) or a culture thereof.

The composition may be for oral administration, feed, feed additives, food, or food additives.

In this respect, the present invention provides a composition for oral administration, feed, feed additives, food, or food additives, including Dunaliella salina OH214 (KCTC 14434BP) and/or a culture thereof.

The composition may be for enhancing or maintaining eye health; preventing or ameliorating macular degeneration; preventing or ameliorating a decline in eye function; ameliorating or preventing retinal damage; maintaining retinal health; reducing the risk of developing macular degeneration; or preventing or ameliorating vision loss.

Furthermore, the present invention provides a method for producing a pigment, the method including culturing Dunaliella salina OH214 (KCTC 14434BP).

The method for producing a pigment may further include extracting a pigment from Dunaliella salina OH214 (KCTC 14434BP) and/or a culture thereof.

The pigment may include one or more selected from the group consisting of lutein, zeaxanthin and β-carotene.

Further, the present invention provides a method for producing a food or feed raw material, the method including culturing Dunaliella salina OH214 (KCTC 14434BP).

### Advantageous Effects

When the Dunaliella salina OH214 strain of the present invention is used, a macular pigment can be efficiently produced and supplied at an industrial level because a large amount of lutein can be produced with a small amount of energy. The composition of the present invention can be applied as a raw material for foods, health functional foods, and pharmaceuticals, containing the lutein pigment. Further, the strain of the present invention is a species that grows in Korea, and in consideration of the physiological characteristics of Dunaliella salina, which is a euryhaline microalga, and the geographical characteristics of Korea, which is surrounded by the sea on three sides, it is also expected to reduce costs and develop related industries because seawater can be used as a culture medium.

### DESCRIPTION OF DRAWINGS

FIG. 1 is a set of micrographs of the Dunaliella salina OH214 strain of the present invention, Dunaliella salina CCAP19/18, and Dunaliella tertiolecta CCAP19/42 (400× magnification). The black line (bar) in the photos denotes 10 μm.

FIG. 2A illustrates the results of a phylogenetic analysis using 18S rRNA, and the scale bar denotes a 2% difference in nucleic acid sequences.

FIG. 2B illustrates the results of a phylogenetic analysis using a sequence containing ITS, and the scale bar denotes a 5% difference in nucleic acid sequences.

FIGS. 3A and 3B are a graph showing the lutein content per culture solution (A) and the lutein content per cell (B) of the Dunaliella salina OH214 strain of the present invention according to the NaCl concentration of the culture medium. The horizontal axis denotes the NaCl concentration (mM) of the culture medium, and the vertical axis denotes the lutein content per liter (mg/L) and the lutein content per cell (pg/cell), respectively.

FIG. 4A is a graph showing the lutein content per culture solution of the Dunaliella salina strain OH214 of the present invention according to illuminance. The horizontal axis denotes the light intensity (units: μmol photons/m² s) treated with the culture medium, and the vertical axis denotes the lutein content (mg/L) per liter of the culture medium.

FIG. 4B is a graph showing the lutein content per cell of the Dunaliella salina strain OH214 of the present invention according to illuminance. The horizontal axis denotes the light intensity (units: μmol photons/m² s) treated with the culture medium, and the vertical axis denotes the lutein content (mg/L) per cell.

FIG. 5 is a graph showing the cell number increase patterns of the Dunaliella salina OH214 strain of the present invention, Dunaliella salina CCAP19/18, and Dunaliella tertiolecta CCAP19/42.

FIGS. 6A and 6B are a graph (A) of the lutein content (mg) per dry weight (g) of the Dunaliella salina OH214 strain of the present invention and the control strains Dunaliella salina CCAP19/18 and Dunaliella tertiolecta CCAP19/42, and a graph (B) in which biomass was measured by the dry weight (g) of each strain per liter (L) of the culture solution.

FIGS. 7A and 7B are a set of graphs comparing the amounts of lutein contained in the Dunaliella salina OH214 strain of the present invention and the control strains Dunaliella salina CCAP19/18 and Dunaliella tertiolecta CCAP19/42: (A) content (mg) of lutein per liter at a light intensity of 200 μmol photons/m² s; and (B) content (pg) of lutein per cell at a light intensity of 200 μmol photons/m² s.

FIGS. 8A and 8B are graphs comparing the amounts of lutein contained in the Dunaliella salina OH214 strain of the invention and the control strain Dunaliella salina CCAP19/18: (A) content (mg) of lutein per liter at a light intensity of 1000 μmol photons/m² s; and (B) content (pg) of lutein per cell at a light intensity of 1000 μmol photons/m² s.

FIGS. 9A and 9B are a set of graphs comparing the amounts of zeaxanthin contained in the Dunaliella salina OH214 strain of the present invention and the control strains Dunaliella salina CCAP19/18 and Dunaliella tertiolecta CCAP19/42: (A) content (mg) of zeaxanthin per liter at a light intensity of 200 μmol photons/m² s; and (B) content (pg) of zeaxanthin per cell at a light intensity of 200 μmol photons/m² s.

FIGS. 10A and 10B a set of graphs comparing the amounts of macular pigments (lutein and zeaxanthin) contained in the Dunaliella salina OH214 strain of the present invention and the control strains Dunaliella salina CCAP19/18 and Dunaliella tertiolecta CCAP19/42: (A) total content (mg) of lutein and zeaxanthin per liter at a light intensity of 200 μmol photons/m² s; and (B) total content (pg) of lutein and zeaxanthin per cell at a light intensity of 200 μmol photons/m² s.

FIG. 11 is an HPLC analysis graph showing the pigment profile of the Dunaliella salina OH214 strain of the present invention: 1: neoxanthin, 2: violaxanthin, 3: antheraxanthin, 4: lutein, 5: zeaxanthin, 6: chlorophyll b, 7: chlorophyll a, 8: β-carotene.

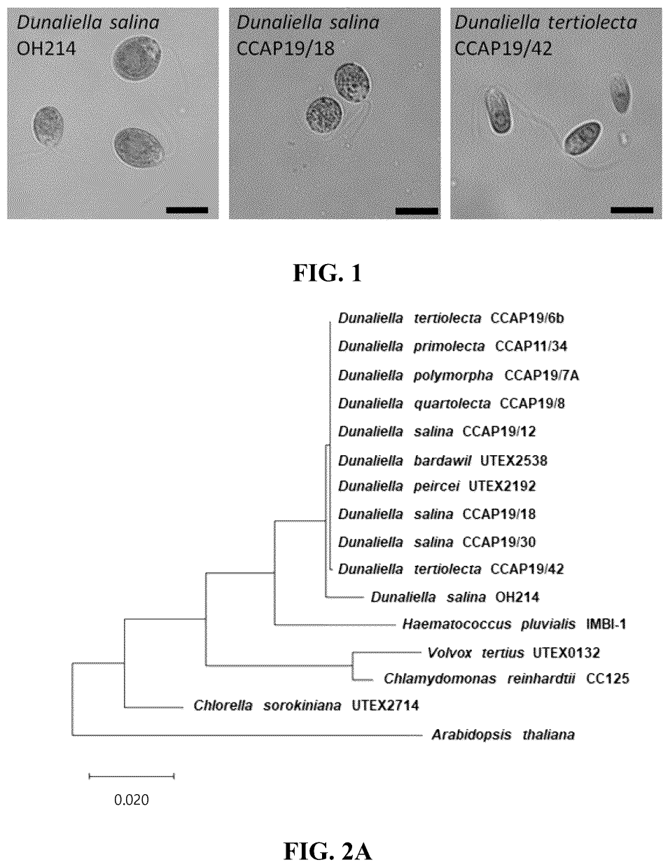

### MODES OF THE INVENTION

Hereinafter, the present disclosure will be described in more detail.

However, the present invention may be modified into various forms and may have various forms, so that specific examples and descriptions set forth below are included merely for aiding the understanding of the present invention and are not intended to limit the present invention to a specific disclosure form. It should be understood that the scope of the present invention includes all the modifications, equivalents, and replacements falling within the spirit and technical scope of the present invention.

The present invention relates to Dunaliella salina OH214 (KCTC 14434BP).

Dunaliella salina is a unicellular marine microalga and is known to be able to survive under various salinity conditions (0.05 to 5.5 M NaCl). Dunaliella salina is a carotenogenic species and is known to produce large amounts of pigments, generally β-carotene, when exposed to stressful environments such as high salt, strong light or high temperature. When exposed to strong light, Dunaliella salina plays a role in protecting photosynthetic organs by accumulating a large amount of β-carotene in carotenoid globules inside the thylakoids of chloroplasts to block strong light. Due to these characteristics, it is known that Dunaliella salina is able to be used as a source of β-carotene.

Dunaliella salina OH214 (KCTC 14434BP), which is newly identified in the present invention, is a domestic native microalga isolated from a salt farm (Geumhong Salt Farm) in the West Sea (Yeongjong Island), Korea, and was identified as Dunaliella salina through a phylogenetic analysis using 18S rRNA and ITS sequence analysis, and was named OH214.

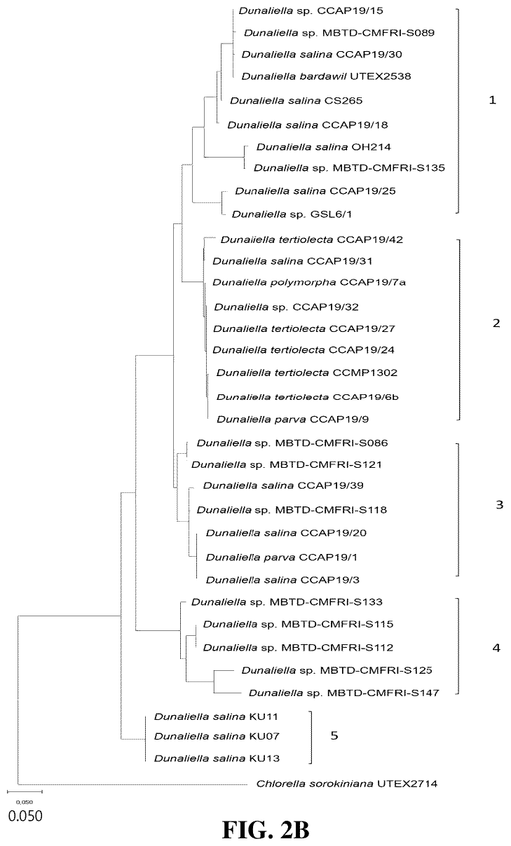

Specifically, genomes were extracted using ITS1 (TCCGGTGAACCTGCG: SEQ ID NO: 1) and ITS4 (TCCGCTTATTGATGC: SEQ ID NO: 2) as primers to amplify the base sequence, and the sequences were compared (aligned) using BioEdit, and shown as a phylogenetic tree diagram using the MAGA-X program (FIGS. 2A and 2B).

Through an exemplary embodiment of the present invention, the Dunaliella salina OH214 strain includes an 18s rDNA base sequence represented by SEQ ID NO: 3.

The Dunaliella salina OH214 strain was deposited with the Korea Research Institute of Bioscience and Biotechnology Korean Collection for Type Cultures (KCTC) on Jan. 5, 2021, and was given the accession number of KCTC 14434BP on Jan. 5, 2021.

The Dunaliella salina OH214 strain of the present invention has an excellent ability to produce pigments, and specifically, it has an extremely excellent ability to produce lutein compared to the genus Dunaliella (sp.) strains in the related art.

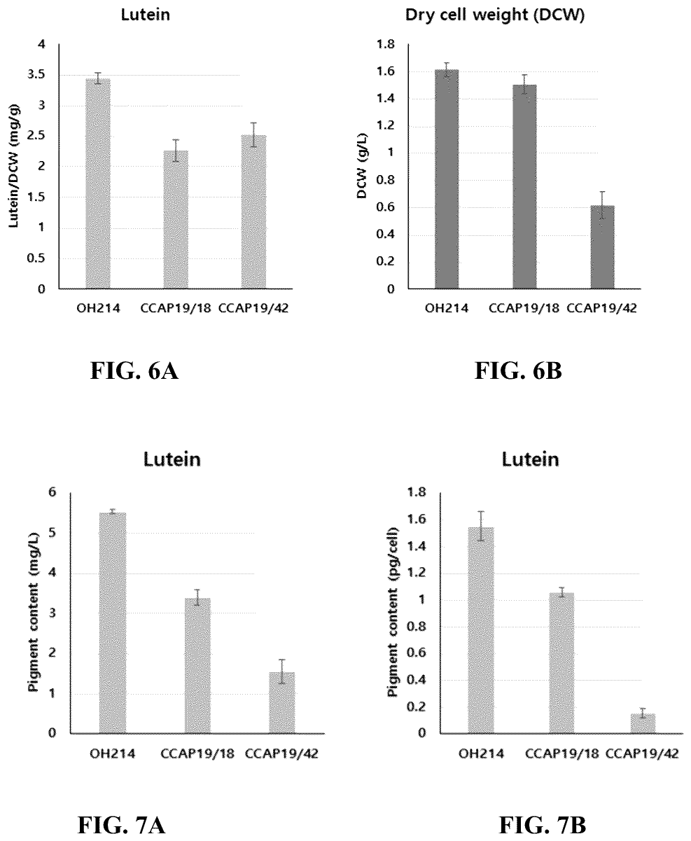

Through an exemplary embodiment of the present invention, the ability of strains to produce lutein was confirmed under conditions of high light intensity (1000 μmol photons/m² s) and low light intensity (200 μmol photons/m² s), and as a result, it was confirmed that under both conditions, the Dunaliella salina OH214 strain of the present invention has remarkably high biomass content, lutein content per dry weight, lutein content per 1 L of culture solution, and lutein content per cell compared to a control strain Dunaliella salina CCAP19/18 or Dunaliella tertiolecta CCAP19/42 (FIGS. 6A, 6B, 7A, 7B, 8A and 8B).

In addition, the Dunaliella salina OH214 strain of the present invention has the ability to produce zeaxanthin and/or β-carotene in addition to lutein.

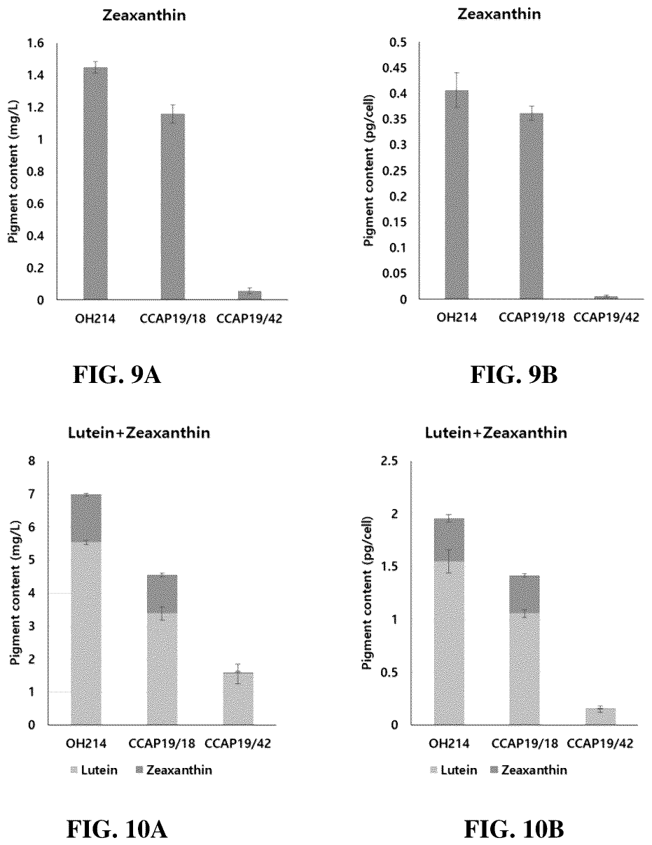

Through an exemplary embodiment of the present invention, as a result of confirming the ability to produce zeaxanthin among strains, it was confirmed that the Dunaliella salina OH214 strain of the present invention has high zeaxanthin content per 1 L of culture solution and zeaxanthin content per cell compared to the control strain Dunaliella salina CCAP19/18 or Dunaliella tertiolecta CCAP19/42 (FIGS. 9A and 9B), and that the total content of lutein and zeaxanthin is also excellent compared to the two control strains (FIGS. 10A and 10B).

Furthermore, as a result of confirming a pigment profile in an exemplary embodiment of the present invention, it was confirmed that the content of β-carotene in the pigment was high, and that the Dunaliella salina OH214 strain of the present invention could also produce additional pigments such as neoxanthin, violaxanthin, antheraxanthin, chlorophyll a and chlorophyll b together.

Therefore, the Dunaliella salina OH214 strain of the present invention has a significantly higher ability to produce lutein and zeaxanthin than the genus Dunaliella microalgae in the related art, and thus, may be effectively used as a microalga for producing xanthophyll.

The Dunaliella salina OH214 strain of the present invention can survive in dim light, and may be cultured under light intensity conditions, specifically within a range of 10 to 2,000 μmol photons/m² s.

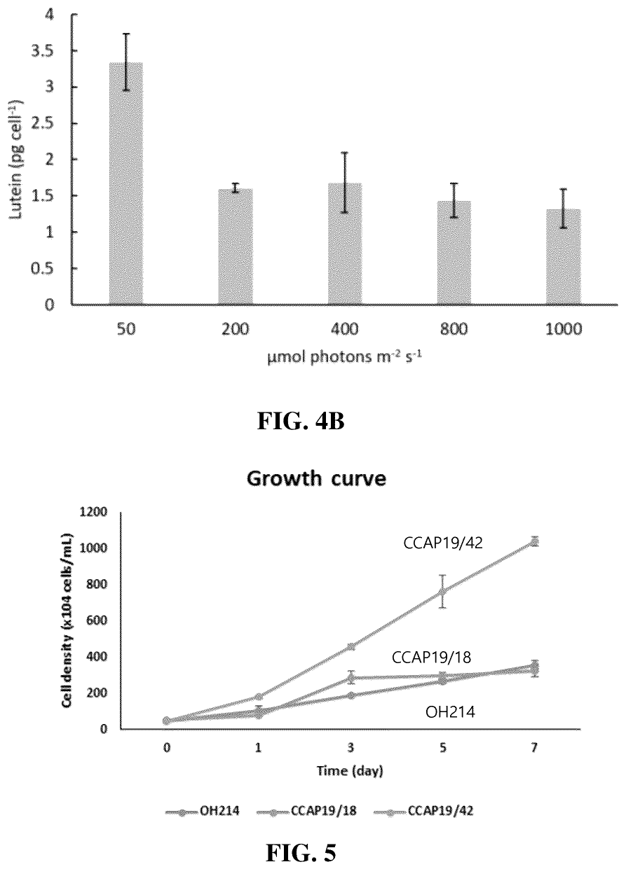

In particular, the Dunaliella salina OH214 strain of the present invention has an advantage in terms of industrial utilization in that it has an excellent ability to produce lutein even under dim light conditions. For strains that increase pigment or biomass production under high light intensity conditions, the value of the culture product relative to the energy input may be lowered, and there are limitations to high light dosing, so that pigment production may not be substantially smooth. However, the Dunaliella salina OH214 strain of the present invention has an advantage in that lutein can be produced without requiring much energy because the lutein content per cell can be maximally obtained under low light conditions of 50 μmol photons/m² s and the lutein content per culture solution is excellent at 200 μmol photons/m² s or more (FIGS. 4A and 4B).

The Dunaliella salina OH214 strain may grow appropriately in typical growth environments (light intensity, temperature, salinity conditions, and the like) for the genus Dunaliella microalgae. Further, the Dunaliella salina OH214 strain has an excellent ability to accumulate lutein even at low light intensity (FIGS. 4A and 4B), and thus, may be effectively used industrially as a microorganism that produces a xanthophyll pigment.

The Dunaliella salina OH214 strain may be cultured in a seawater environment, specifically in a culture medium including seawater. The Dunaliella salina OH214 strain of the present invention may be cultured under NaCl concentration conditions of 0.05 M to 5.5 M based on the concentration of NaCl. The culture medium may further include Tris in addition to NaCl.

The culture medium of the Dunaliella salina strain OH214 of the present invention may include salt at a concentration of 0.1 M to 2.0 M NaCl. In this case, since the growth rate of the Dunaliella salina OH214 strain of the present invention is excellent, the production efficiency of pigments including lutein may be significantly increased.

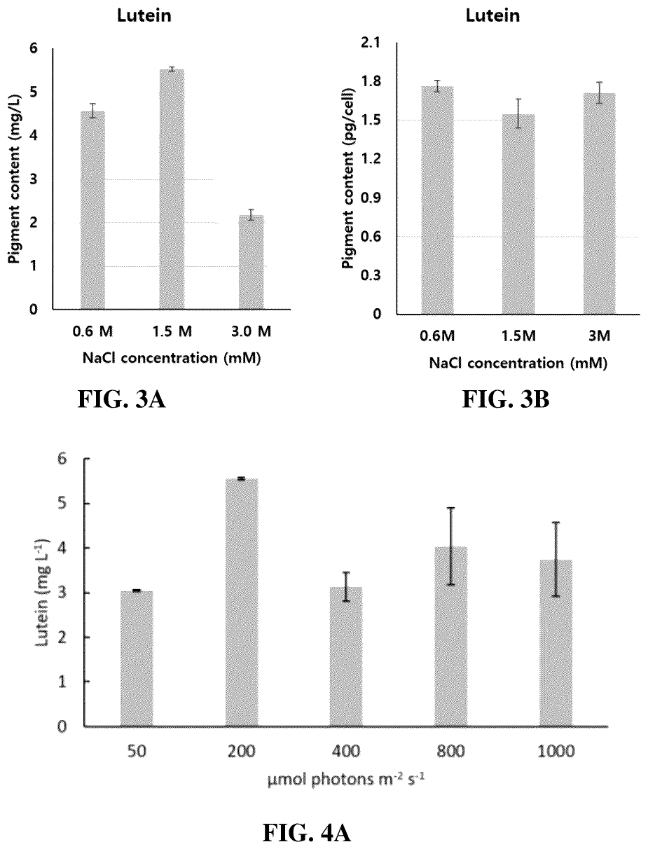

In an exemplary embodiment of the present invention, the Dunaliella salina OH214 strain has an excellent ability to produce lutein regardless of NaCl concentration (FIGS. 3A and 3B), and it was confirmed through an exemplary embodiment that the growth rate of the strain is the best under the 1.5 M NaCl concentration condition.

The culture medium contains nutrients required by a microorganism to be cultured, that is, a cultured organism, in order to culture a specific microorganism, and may be a medium in which a material for a special purpose is additionally added and mixed. The medium also refers to a culture medium or a culture solution, and is a concept encompassing all of natural media, synthetic media or selective media. The Dunaliella salina OH214 strain may be cultured according to a typical culture method. In an exemplary embodiment, it was confirmed that in the culture solution shown in Table 3, the Dunaliella salina OH214 strain of the present invention had an excellent ability to produce lutein.

A pH of the culture medium is not particularly limited as long as the pH is within a range enabling the genus Dunaliella microalgae to survive and grow, and the genus Dunaliella microalgae can survive, for example, at a pH of 6 or more, specifically at a PH of 7 to a pH of 9, and may have an optimal growth rate at a pH of 8.0 or more and a pH of less than 9.0.

The Dunaliella salina OH214 strain of the present invention may accumulate a high content of pigments, particularly xanthophyll-based pigments, in cells thereof, and thus, may be effectively used as a pigment supply source for foods, feeds, pharmaceuticals, and the like.

In this respect, the present invention provides a culture of Dunaliella salina OH214 (KCTC 14434BP).

As used herein, the “culture” refers to a medium in which a specific microorganism is cultured, that is, a medium after culturing, and the above-described culture is meant to include anything that includes Dunaliella salina OH214 or that has been sterilized through filtration after culturing Dunaliella salina OH214 of the present invention. In addition, the “culture” is meant to include all of a concentrate of a culture obtained by concentrating the culture medium after culturing, a dry product of a culture obtained by processing such as drying, or an extract thereof. In this respect, the culture of the invention may include Dunaliella salina OH214, byproducts of Dunaliella salina OH214 and/or pigments produced by Dunaliella salina OH214.

The formulation of the culture is not limited, and may be, for example, in the form of a liquid, solid or gel.

In the present specification, the “medium” contains nutrients required by a microorganism to be cultured, that is, a cultured organism, in order to culture a specific microorganism, and may be a medium in which a material for a special purpose is additionally added and mixed. The medium also refers to a culture medium or a culture solution, and is a concept encompassing all of natural media, synthetic media or selective media. A pH of the medium may be in a range in which Dunaliella salina OH214 can grow, and may be a pH of 6 or more as an example, and preferably a pH of 7 to 9.

As the medium composition of the present invention, any medium may be included and utilized in the present invention as long as it is a medium composition in which the genus Dunaliella (sp.), particularly Dunaliella salina, can grow, and may be, for example, a medium having the composition shown in Table 3.

Furthermore, the present invention provides a composition including Dunaliella salina OH214 (KCTC 14434BP) and/or a culture thereof.

The composition may be used to improve the health of humans and animals.

Since the Dunaliella salina OH214 of the present invention has characteristics of producing a xanthophyll-based pigment including zeaxanthin and lutein and accumulating the pigment in the body, in this respect, the composition may be a pigment composition or a xanthophyll pigment composition.

The composition may be for oral administration in that the Dunaliella salina OH214 strain or a pigment produced therefrom may be used as a raw material for food, feed, health functional food, pharmaceuticals, and the like, and may be supplied orally by being included in food, pharmaceuticals, feed, or the like.

Further, since the composition can be added to food or feed in order to achieve a special purpose, in this respect, it may be a food composition, a food additive composition, a feed composition or a feed additive composition.

When the composition is used in feed or food, physical health may be maintained or strengthened by a xanthophyll pigment, particularly, zeaxanthin and lutein produced by Dunaliella salina OH214 and accumulated in the cells.

Specifically, zeaxanthin and lutein may prevent or alleviate degeneration of the macula, and the like as a macular pigment, and thus are effective for preventing or alleviating eye disorders related to macular degeneration. More specifically, since zeaxanthin and lutein have effects of strengthening or maintaining eye health; preventing or alleviating macular degeneration; preventing or alleviating a decline in eye function; alleviating or preventing damage to the retina; maintaining retinal health; reducing the risk of developing macular degeneration; or preventing or alleviating vision loss, the feed or food composition may be used to prevent or alleviate the above symptoms or for the above effects. The decline in eye function includes a decline in eye function caused by external environments such as ultraviolet rays or excessive use of monitors, or a decline in eye function caused by aging, and the like.

The composition of the present invention may provide the aforementioned effects by supplying a macular pigment to prevent or alleviate intraretinal damage induced by ultraviolet rays or reactive oxygen species.

In the present invention, “for an additive” includes any food composition as long as the food composition is a composition in which ingredients other than the main ingredient are added to food or feed, and a specific example thereof may be an active material having functionality in food or feed or a food additive defined by the Ministry of Food and Drug Safety of the Republic of Korea, which are added for coloring, preservation, and the like in a processed food.

The food may be a health functional food. More specifically, the food may be a food functional food for eye health. In this respect, the present invention provides a composition for a health functional food, including Dunaliella salina OH214 (KCTC 14434BP) and/or a culture thereof.

The composition for a food, food additives, feed or feed additives may further include other active ingredients within a range that does not impair the activity of the Dunaliella salina OH214 (KCTC 14434BP) of the present invention and/or a culture thereof. Further, it is possible to further include an additional ingredient such as a carrier.

In the present invention, a composition for feed may be prepared in the form of fermented feed, blended feed, a pellet, silage, and the like.

The fermented feed may include the Dunaliella salina OH214 strain of the present invention, dried microbial cells of the strain, a culture thereof, or an extract thereof. In addition, the fermented feed may additionally include various microorganisms or enzymes. The blended feed may be prepared by including various types of general feed, the Dunaliella salina OH214 strain of the present invention, dried microbial cells of the mutant, a culture thereof, and an extract thereof and mixing the mixture. A feed in the form of a pellet may be prepared by formulating the fermented feed or blended feed with a pellet machine. The silage may be prepared by mixing soilage feed with the Dunaliella salina OH214 strain, dried microbial cells of the mutant, a culture thereof and/or an extract thereof, but the use of the composition of the present invention is not limited thereto.

The composition may be mixed with a carrier and a flavoring typically used in the food or pharmaceutical field and may be prepared and administered in the form of a tablet, a troche, a capsule, an elixir, a syrup, a powder, a suspension, a granule, or the like. As the carrier, it is possible to use a binder, a lubricant, a disintegrating agent, an excipient, a solubilizing agent, a dispersing agent, a stabilizing agent, a suspending agent, and the like. As an administration method, an oral, parenteral, or application method may be used, but oral administration is preferred. In addition, the administered dose may be appropriately selected depending on the absorption degree, the inactivation rate and the excretion rate of an active ingredient in the body, and age, gender, condition, and the like of a person to receive the administration. A pH of the composition can be easily changed depending on the manufacturing conditions and the like of medicine, food, and the like under which the composition is used.

The composition may include any one selected from the group consisting of Dunaliella salina OH214 (KCTC 14434BP), a culture thereof, a dry product thereof, and an extract thereof in an amount of 0.001 to 99.99 wt %, preferably 0.1 to 99 wt %, based on the total weight of the composition, and the content of an active ingredient may be appropriately adjusted depending on the method for using the composition and the purpose of using the composition.

Furthermore, the composition may be composed only of those selected from Dunaliella salina OH214 (KCTC 14434BP), a culture thereof, a dry product thereof, and an extract thereof.

The Dunaliella salina OH214 (KCTC 14434BP) may be included as is or in a dried form in the composition, and a culture thereof may be included in a concentrated or dried form in the composition. Further, the dry product refers to a dried form of the Dunaliella salina OH214 of the present invention or the culture thereof, and may be, for example, in the form of a powder prepared by lyophilization, and the like. As for the drying method, any drying method typically used in the technical field of the present invention can be used without limitation.

Further, the extract refers to an extract obtained by extracting a product from the Dunaliella salina OH214 strain of the present invention, a culture solution thereof, or a dry product thereof, and includes an extract using a solvent, and the like, and an extract obtained by crushing the strain of the present invention. Specifically, the extract may be obtained by extracting and isolating pigments accumulated in the cells of the Dunaliella salina OH214 (KCTC 14434BP) of the present invention using a physical or chemical method.

The extraction procedure may be carried out by a typical method, and as an example, a target pigment may be extracted by adding an extraction solvent to the Dunaliella salina OH214 (KCTC 14434BP) of the present invention, homogenizing the resulting mixture, and then crushing the microbial cells. After the extraction, a crushed material of the strain may be removed through centrifugation, and the extraction solvent may be removed by a method such as distillation under reduced pressure. In addition, the extraction procedure may further include a typical purification process. The aforementioned pigment has the property of being insoluble in water, and thus can be more easily extracted from the strain of the present invention.

Since the Dunaliella salina OH214 strain of the present invention has an excellent ability to produce xanthophyll, particularly zeaxanthin at low light intensity, a compound including the Dunaliella salina OH214 strain and a byproduct thereof has effects of improving body activity, maintaining body functionality, and preventing a decline in body functionality. Specifically, since the xanthophyll pigment is known to have an effect of suppressing macular degeneration, antioxidant and anticancer effects, and the like, the composition of the present invention may be used as a raw material included in food, a pharmaceutical, feed, and the like for the purpose of maintaining physical health, specifically, maintaining body functionality with which the xanthophyll pigment is associated, preventing a decline in body functionality, or improving body functionality.

Furthermore, the present invention provides a method for producing a pigment, the method including culturing Dunaliella salina OH214 (KCTC 14434BP).

Further, the present invention provides a method for producing a food or feed raw material, the method including culturing Dunaliella salina OH214 (KCTC 14434BP).

When the Dunaliella salina OH214 (KCTC 14434BP) of the present invention is used, it is possible to increase the amount of xanthophyll pigments accumulated in cultured microalgae, so that it is possible to efficiently supply raw materials for industrial use, and the like.

The pigment may include one or more selected from the group consisting of lutein, zeaxanthin, and β-carotene, preferably lutein; or may include lutein and one or more of zeaxanthin or β-carotene.

In addition, the production method may further include isolating the Dunaliella salina OH214 (KCTC 14434BP) of the present invention from the culture after the culturing step. The isolated Dunaliella salina OH214 cells may be further subjected to a processing step including drying.

Furthermore, the production method may further include extracting a pigment from Dunaliella salina OH214 (KCTC 14434BP) and/or a culture thereof.

The culturing may be performed in a medium under salinity conditions of 0.05 M to 5.5 M NaCl based on the concentration of NaCl. Preferably, the culturing may be performed under a concentration condition of 0.5 M NaCl to 2.0 M NaCl, and in this case, the strain of the present invention has an excellent growth rate, and thus, pigment production efficiency may be improved.

Further, the culturing may be performed under dim light conditions, specifically, under light intensity conditions within a range of 10 to 2,000 μmol photons/m² s. Since the Dunaliella salina OH214 strain of the present invention has an excellent ability to produce a pigment, particularly lutein, even at low light intensity, the strain may achieve excellent accumulation of a xanthophyll pigment without inputting high light energy, which is industrially meaningful.

The extraction may be carried out by a typical method for extracting a pigment from microorganisms, and examples thereof include an enzyme method, ultrasonic extraction, a mechanical extraction method, and the like, and are not limited thereto.

The production method may further include, in addition to the culturing step, a concentrating step of increasing the content of the strain after the culturing and a drying step of drying the strain subjected to the concentrating step by further reducing moisture in the strain. However, the concentrating step or the drying step is not necessarily needed, and may be generally carried out by using a concentrating and drying method, and a machine typically used in the field to which the present invention belongs.

The preparation method may further include a purification step after the extracting step, and the purification step may be carried out by a typical purification method in the field to which the present invention belongs.

A pigment produced through the concentrating or drying step may be used as a raw material for food, health functional food, cosmetics, feed, pharmaceuticals, or the like.

The method for producing the pigment may be carried out by adopting other methods within a range that does not impair the effects of the present invention.

The contents described above regarding the strain of the present invention, a culture thereof, and a composition including the same may also be applied mutatis mutandis to the production method of the present invention.

### EXAMPLES

Hereinafter, the present invention will be described in detail through Preparation Examples and Experimental Examples. The following Preparation Examples and Examples are only for exemplifying the present invention, and the scope of the present invention is not limited thereto.

### Example 1. Screening and Identification of Strain

1-1. Isolation of Strain

Samples were collected from Yeongjong-do salt fields in the West Sea, and the strain was isolated using the Dunaliella salina's characteristic of red accumulation of β-carotene at high light intensity.

First, the collected seawater was diluted to various concentrations and spread on 1% agarose containing 3 M salt, and the agarose was incubated under the conditions of a high light intensity of 800 μmol photons/m² s and 25 to 26° C. for two weeks. Thereafter, it was confirmed that red colonies were formed, and the red colonies were aseptically streaked again and inoculated into a 1.5 M D medium (Sathasivam et al. 2014) and isolated as a single species.

1-2. Identification of Strain

For phylogenetic analysis, the cells cultured in the 1.5 M D medium were centrifuged at 13,000 rpm, and the resulting cell pellet was used for genomic DNA extraction using the CTAB method. Genomic DNA (100 ng) was mixed with KOD PCR master mix (TOYOBO) and the resulting mixture was amplified by PCR. The specific conditions are as follows: 34 cycles of an amplification step consisting of a pre-denaturation step (95° C., 3 minutes) and a denaturation step (98° C., 10 seconds), an annealing step (55° C., 5 seconds), and an extension step (68° C., 10 seconds). For amplification, 18S rRNA, an internal transcribed spacer (ITS), and universal primers were used; ITS1 (TCCGTAGGTGAACCTGCGG, SEQ ID NO: 1) and ITS4 (TCCTCCGCTTATTGATATGC, SEQ ID NO: 2) were each used at a concentration of 10 μmol. Amplified PCR products were purified using a DokDo-Prep PCR purification kit (ELPISBIOTECH. INC.). The purified PCR products were electrophoresed on a 1.5% agarose gel to confirm the generation of accurate PCR products, and comparison of the sequence information of the selected new species was performed through a BLAST search in the National Center for Biotechnology Information (NCBI) database. The 18S rRNA or ITS sequence of a species closely related to the single species was downloaded and sequence-aligned using the BioEdit program. Then, a phylogenetic tree was drawn using the MAGA-X program to identify the isolated strains.

As targets for sequence comparison obtained from the NCBI, the 18S rRNA sequence information is shown in the following Table 1, and the ITS sequence information is shown in Table 2.

TABLE 1

| Species | Isolation source | Accession No. |
|---|---|---|
| Dunaliella salina OH214 | Yeongjong Island, Korea | Present invention |
| Arabidopsis thaliana | Qualifiers | X16077 |
| Volvox tertius UTEX0132 | Queen's Ditch, Cambridge, | FJ610144 |
| | England | |
| Chlamydomonas | Qualifiers | KR092109 |
| reinhardtil_CC125 | | |
| Haematococcus | Qualifiers | KU193764 |
| pluvialis_IMBI-1 | | |
| Dunaliella | Oslo fjord, Norway | DQ009773 |
| tertiloecta_UTEX999 | | |
| Dunaliella | Marine, Israel | Unpublished |
| tertiolecta_CCAP19/42 | | |
| Dunaliella | Hutt Lagoon, Western | EF473745 |
| salina_CCAP19/18 | Australia | |
| Dunaliella | Qualifiers | DQ447648 |
| salina_CCAP19/30 | | |
| Dunaliella | Lake Marina, USA | DQ009778 |
| peircei_UTEX2192 | | |
| Dunaliella | Bardawil Lagoon, Israel | DQ009777 |
| bardawil_UTEX2538 | | |
| Dunaliella | Brackish, North Sinai Israel | KJ756842 |
| salina_CCAP19/12 | | |
| Dunaliella | Marine, Hampshire UK | KJ756824 |
| quartolecta_CCAP19/8 | | |
| Dunaliella | Brackish, Isle of Wight UK | KJ756821 |
| polymorpha_CCAP19/7A | | |
| Dunaliella | Brackish, Oslo Fjord, | KJ756820 |
| tertiolecta_CCAP19/6b | Norway | |
| Dunaliella | Marine, Devon, England, | KJ756819 |
| primolecta_CCAP11/34 | UK | |
| Chlorella | Qualifiers | LK021940 |
| sorokinianan_UTEX2714 | | |

TABLE 2

| Species | Isolation source | Accession No. |
|---|---|---|
| Dunaliella salina OH214 | Yeongjong Island, Korea | Present invention |
| Dunaliella salina | Hutt Lagoon, Western | EF473746 |
| CCAP19/18 | Australia | |
| Dunaliella salina | Qualifiers | KJ094632 |
| CCAP19/30 | | |
| Dunaliella salina | Qualifiers | KJ094624 |
| CCAP19/20 | | |
| Dunaliella bardawil | Bardawil Lagoon, Israel | DQ377085 |
| UTEX2538 | | |
| Dunaliella tertilolecta | Halifax, Canada | KJ094631 |
| CCAP19/27 | | |
| Dunaliella tertilolecta | Qualifiers | KJ094628 |
| CCAP19/24 | | |
| Dunaliella tertilolecta | Marine, Israel | Unpublished |
| CCAP19/42 | | |
| Dunaliella tertilolecta | Qualifiers | DQ377096 |
| CCMP1302 | | |
| Dunaliella tertilolecta | Brackish, Oslo Fjord, | KJ094612 |
| CCAP19/6b | Norway | |
| Dunaliella salina | Qualifiers | KJ094629 |
| CCAP19/25 | | |
| Dunaliella parva | Qualifiers | KJ094607 |
| CCAP19/1 | | |
| Dunaliella parva | Salt marsh, Essex, | KJ094617 |
| CCAP19/9 | England, UK | |
| Dunaliella polymorpha | Qualifiers | KJ094613 |
| CCAP19/7a | | |
| Dunaliella sp. CCAP19/15 | Brackish, North Sinai, Israel | KJ094621 |
| Dunaliella sp. CCAP19/32 | salt deposit, California, USA | KJ094634 |
| Dunaliella salina | Qualifiers | KJ094609 |
| CCAP19/3 | | |
| Dunaliella sp. CCAP19/39 | sea salt, Gran Canaria, Spain | KJ094637 |
| Dunaliella salina | Qualifiers | KJ094633 |
| CCAP19/31 | | |
| Dunaliella salina CS265 | Qualifiers | JN797804 |
| Dunaliella sp. MBTD- | Sea water, Calicut, Kerala | JN797802 |
| CMFRI-S135 | | |
| Dunaliella sp. MBTD- | Salt pan, Chennai, TN (EC) | JN797806 |
| CMFRI-S089 | | |
| Dunaliella sp. MBTD- | Salt pan, Nellore, AP (EC) | JN797808 |
| CMFRI-S118 | | |
| Dunaliella sp. MBTD- | Salt pan, Tuticorin, TN (EC) | JN797805 |
| CMFRI-S086 | | |
| Dunaliella sp. MBTD- | Pulicat salt lake, AP (EC) | JN797809 |
| CMFRI-S121 | | |
| Dunaliella sp. MBTD- | Salt pan, Chennai, TN (EC) | JN797807 |
| CMFRI-S115 | | |
| Dunaliella sp. MBTD- | Salt pan, Ribandar, Goa | JN797799 |
| CMFRI-S122 | (WC) | |
| Dunaliella sp. MBTD- | Salt pan, Kutch, Gujarat | JN797801 |
| CMFRI-S133 | (WC) | |
| Dunaliella sp. MBTD- | Salt pan, Pilar, Gor (WC) | JN797800 |
| CMFRI-S125 | | |
| Dunaliella sp. MBTD- | Salt pan, Kutch, Gujarat | JN797803 |
| CMFRI-S147 | (WC) | |
| Dunaliella salina isolate | Zaranik, Noth Sinai | JX220893 |
| SEA003 | | |
| Dunaliella salina KU07 | Northeastern part, Thailand | KF825548 |
| Dunaliella salina KU11 | Northeastern part, Thailand | KF825549 |
| Dunaliella salina KU13 | Northeastern part, Thailand | KF825547 |
| Dunaliella sp. GSL6/1 | Great Salt Lake, U.S.A. | MG952975 |
| Chlorella sorokiniana | Qualifiers | LK021940 |
| UTEX2714 | | |

As shown in FIG. 2A, it was confirmed that as a result of analysis using the 18S rRNA sequence, the isolated strain of the present invention is very close to the strain of the genus Dunaliella (sp.), and thus belongs to the genus Dunaliella (sp.). In addition, as shown in FIG. 2B, it was confirmed that as a result of phylogenetic analysis using a sequence including ITS1, 5.8S rRNA, and ITS2, the isolated strain of the present invention belongs to the same Glade as Dunaliella salina CCAP19/18 and UTEX2538, which are known as carotenoid-accumulating strains. According to the analysis results, the isolated and identified strain was named Dunaliella salina OH214 and was given the accession number KCTC 14434BP by the Korea Research Institute of Bioscience and Biotechnology Korean Collection for Type Cultures (KCTC) on Jan. 5, 2021. The Dunaliella salina OH214 strain includes an 18s rDNA base sequence represented by SEQ ID NO: 3.

### Example 2. Confirmation of Growth Characteristics and Pigment Content of Strain

2-1. Confirmation of Growth and Pigment Production Ability of Strain According to Salt Concentration of Medium

To compare the cell growth rate and final growth amount of the OH214 strain of the present invention, the growth analysis of the Dunaliella salina OH214 strain was performed at various salt concentrations. The medium composition for seed culture is shown in Table 3 (D medium).

TABLE 3

| | |
|---|---|
| | Concentration in |
| Component | culture solution |

Salt and major components

| NaCl | 1.5M | |
|---|---|---|
| Tris-HCl (pH 7.4) | 40 | mM |
| KNO₃ | 5 | mM |
| MgCl₂ | 4.5 | mM |
| MgSO₄ | 0.5 | mM |
| CaCl₂ | 0.3 | mM |
| K₂HPO₄ | 0.1 | mM |
| FeCl₃ | 0.002 | mM |
| NaEDTA | 0.02 | mM |

Trace components

| | | |
|---|---|---|
| H₃BO₃ | 50 | μM |
| MnCl₂ | 10 | μM |
| ZnSO₄ | 0.8 | μM |
| CuSO₄ | 0.4 | μM |
| NaMoO₄ | 2 | μM |
| NaVO₃ | 1.5 | μM |
| CoCl₂ | 0.2 | μM |

Carbon source

| | |
|---|---|
| NaHCO₃ | 0.025M |
| pH of culture solution | 7.4 |

After inoculation into the medium (D medium) including the components shown in Table 3 at a cell density of 5×10⁵ cells/mL, shaking culture was performed for one week at a temperature of 25 to 26° C. under a light intensity of 200 μmol photons/m² s under the conditions of 0.6 M, 1.5 M, or 3.0 M NaCl, respectively. On each of days 1, 3, 5, and 7 after the start of culture, 0.5 mL of the culture solution was collected, and the number of cells per milliliter was counted under a microscope using a hemocytometer, and a pigment analysis was performed. The pigment analysis was performed specifically according to the following steps. Each day, 500 μL of a sample was aliquoted into 1.5 ml tubes, and the cells were pelleted by centrifugation at 13,000 rpm for 5 minutes. After the supernatant was removed, 450 μL of 100% acetone was added to the tube, and the cell pellet was treated with ultrasonic waves (Branson 5210R-MT Ultrasonic Cleaner, USA) for 5 seconds. To prevent an excessive amount of acetone from being evaporated, 50 μL of distilled water was added to make 90% acetone. The mixture was then filtered through a nylon filter (0.2-μm nylon filter, Whatman), and the supernatant was transferred to a glass vial for high performance liquid chromatography (HPLC) analysis. Pigment analysis was performed using a Shimadzu Prominence HPLC system (LC-20AD, Shimadzu, Japan) equipped with a Waters Spherisorb S5 ODS2 cartridge column. The retention time for each pigment was used as a standard measured with standard pigment samples.

As shown in FIGS. 3A and 3B, it was confirmed that on day 7 of culture, the production amount of lutein (5.5 mg/L) was highest in the medium at a concentration of 1.5 M NaCl. The next highest lutein production amount was 4.5 mg/L at a concentration of 0.6 M NaCl (FIG. 3A). However, as a result of confirming the lutein content per cell, it was confirmed that there was no large difference according to NaCl concentration (FIG. 3B). Through the above results, it can be seen that the Dunaliella salina OH214 strain of the present invention has the ability to produce lutein regardless of the NaCl concentration condition, but the growth rate of the strain was the best at a salt concentration of 1.5 M, resulting in a high lutein content per culture solution.

2-2. Confirmation of Growth and Pigment Production Ability of Strain According to Illuminance

To maintain a seed culture, cells were shake-cultured (100 rpm) in a 500 mL culture flask at a light intensity of 50 μmol photons/m² s and acclimated to each salt medium for 1 week. For experiments, the cells were inoculated at a concentration of 5×10⁵ cells/mL and cultured in an incubator (Photon Systems Instruments, MC 1,000-OD, Czech Republic). As the culture medium, D medium having the composition shown in Table 3 above was used.

The Dunaliella salina OH214 strain of the present invention was cultured by fixing the salt concentration of the culture medium at 1.5 M NaCl and the temperature at 25° C., and varying the light intensity to 50, 200, 400, 800, or 1000 μmol photons/m² s. The content of accumulated lutein was confirmed by harvesting cells on day 7 of culture. The pigment analysis method was performed in the same manner as in Example 2-1.

As shown in FIGS. 4A and 4B, the lutein content per cell was highest under the condition of 50 μmol photons/m² s (3.0 pg/cell), but when the light intensity increased, the lutein content per cell rather decreased. Through this, it can be seen that the strain of the present invention has an excellent ability to produce lutein at low light intensity. However, as shown in FIG. 4A, when the light intensity was 200 μmol photons/m² s, the production amount of lutein per liter of culture solution was highest, so that subsequent experiments were performed under the conditions of 200 μmol photons/m² s.

### Example 3. Comparison of Pigment Production Ability Among Strains

3-1. Comparison of Lutein Production Ability

Through a pigment analysis using HPLC, the lutein production amounts of Dunaliella salina CCAP19/18, Dunaliella tertiolecta CCAP19/42, and Dunaliella salina OH214 were compared. In the same manner as the culture conditions in Example 2, cells were inoculated at 5×10⁵ cells/mL and cultured for 7 days at a light intensity of 200 μmol photons/m² s, and then pigment contents were measured using HPLC. In addition, each strain was cultured for 7 days under the same conditions except for changing the light intensity to 1000 μmol photons/m² s, and the pigment contents were measured using HPLC. The seed strains (D. salina CCAP19/18 and D. salina OH214) grown under the culture conditions described in Example 2 were harvested, the pigment was extracted using 80% acetone, and the centrifuged supernatant was filtered again using a nylon filter, and then injected into an HPLC column for analysis. Other specific pigment analysis conditions were performed as disclosed in Example 2-1.

Furthermore, the biomass of microalgae inoculated and cultured under the same conditions as described above was measured from cells harvested through centrifugation. Cells were harvested from the 15 mL tube by performing centrifugation at 3200 rpm for 5 min and removing the supernatant, and a harvested cell pellet was resuspended and transferred to a pre-weighed 2 mL tube. The pellet was washed three times with D medium and dried overnight using a freeze dryer. Dry weight was measured to determine the biomass of cells.

As shown in FIGS. 6A and 6B, it was confirmed that the Dunaliella salina OH214 strain had the highest dry weight per culture solution, and the lutein content per dry weight was excellent in the Dunaliella salina OH214 strain compared to the other strains. This means that the Dunaliella salina OH214 strain of the present invention has a remarkably excellent ability to produce lutein compared to other strains.

Further, as a result of quantitatively analyzing the lutein content in the strains cultured under 200 μmol photons/m² s light intensity condition using chromatograms, as shown in FIGS. 7A and 7B, it was confirmed that the lutein content per liter of the culture solution was remarkably excellent for the Dunaliella salina OH214 strain compared to the lutein production amounts of Dunaliella salina CCAP19/18 or Dunaliella tertiolecta CCAP19/42 (FIG. 7A). In addition, as a result of confirming the lutein content per cell by harvesting only cells from the culture solution, it was confirmed that the lutein content per cell of the OH214 strain of the present invention was increased by about 40% to about 700% or more compared to that of the control strain Dunaliella salina CCAP19/18 or Dunaliella tertiolecta CCAP19/42 under the same culture conditions and lutein was accumulated (FIG. 7B).

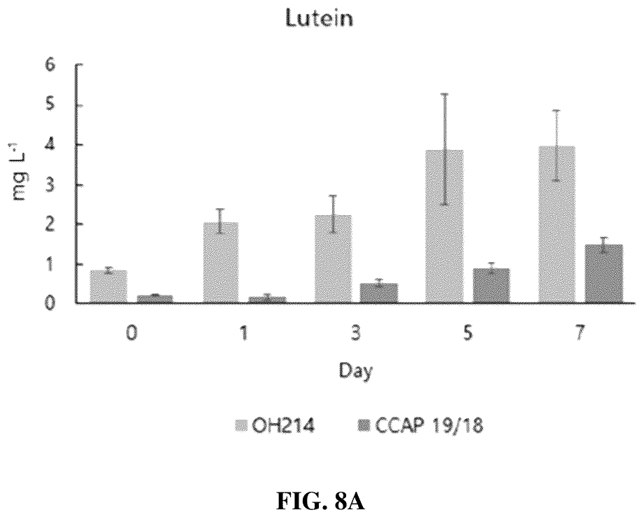

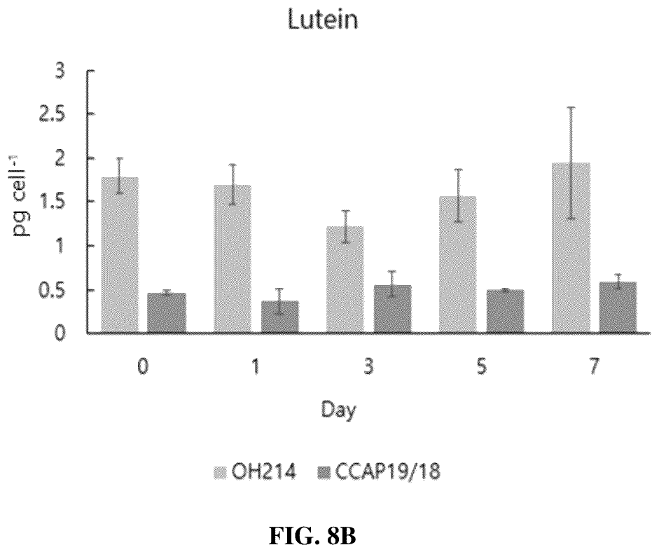

Furthermore, as a result of quantitatively analyzing the lutein content in the strains cultured under the 1000 μmol photons/m² s light intensity condition using chromatograms, as shown in FIGS. 8A and 8B, it was confirmed that during the entire culture period, both the lutein content per liter of culture solution and the lutein content per cell of the Dunaliella salina strain OH214 were 3-fold higher compared to the Dunaliella salina CCAP19/18 strain. Therefore, it can be seen that the Dunaliella salina OH214 strain of the present invention has an excellent ability to produce lutein compared to known strains under both low and high light intensities.

3-2. Comparison of Lutein Production Ability with Dunaliella

The lutein production ability of the microalgae of the present invention was compared with those of other carotenogenic and non-carotenogenic species belonging to the genus Dunaliella (sp.). The cells of Dunaliella salina OH214, Dunaliella salina CCAP19/18, Dunaliella bardawil UTEX2538 and Dunaliella tertiolecta CCAP19/42 were cultured in a 1.5 M D medium under a light intensity of 50 μmol photons/m² s, and cultured at a light intensity of 200 μmol photons/m² s after adjusting the number of cells inoculated to the same level (5×10⁵ cells/mL). After culturing for 1 week, the dry weight of the cells was measured by HPLC every 2 days for 7 days.

3-3. Comparison of Zeaxanthin Production Ability

The ability of Dunaliella salina CCAP19/18, Dunaliella tertiolecta CCAP19/42 and Dunaliella salina OH214 to produce zeaxanthin and other pigments was confirmed under the same conditions and in the same manner as the lutein content analysis.

Culturing and harvesting of the strains were performed under the same conditions and in the same manner as in Example 3-1 above, and specific pigment analysis was performed according to the following method.

In order to isolate the pigment, the total flow rate of the solvent was set at 1.2 mL per minute, and Tris with a pH of 8.0 and acetonitrile were each uniformly decreased from 14% and 84%, respectively to 0% for from the 0th minute to the 15th minute, and methanol and ethyl acetate were increased from 2% to 68% and 32%, respectively up to the 15th minute. Thereafter, the above solvent ratio was maintained for 3 minutes, the ratio of each solvent was returned to the starting ratio for 1 minute, and a post-run was performed while maintaining the ratio as it was for the remaining 6 minutes. LC-20A Prominence manufactured by Shimadzu Company was used as a pump, Watera Spherisorb™ S5 DS1 4.6×250 mm, 5 μm Cartridge Column (Maple St. Milford, Massachusetts, USA) was used as a column, and the temperature of the column was maintained at 40° C. Data was analyzed using a photodiode array detector (SPD-M20A, Shimadzu) as a detector, and the concentrations were obtained using the results in which carotenoid pigments including zeaxanthin and chlorophyll a were detected at 445 nm and 670 nm, respectively.

As shown in FIGS. 9A and 9B, as a result of culturing the cells at a light intensity of 200 μmol photons/m² s for 7 days, it can be confirmed that the Dunaliella salina OH214 strain of the present invention has an excellent ability to produce zeaxanthin compared to Dunaliella salina CCAP19/18 and Dunaliella tertiolecta CCAP19/42.

Further, when the total amounts of lutein and zeaxanthin, which are utilized as macular pigments, were compared (FIGS. 10A and 10B), it could be confirmed that the Dunaliella salina OH214 strain of the present invention had an excellent total content of zeaxanthin and lutein compared to Dunaliella salina CCAP19/18 and Dunaliella tertiolecta CCAP19/42.

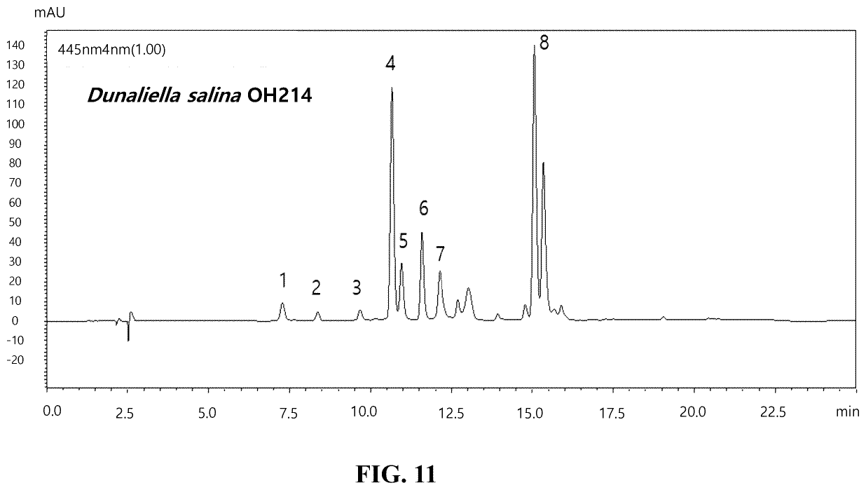

In addition, as shown in FIG. 11, as a result of confirming a pigment profile produced by the Dunaliella salina OH214 strain of the present invention, it can be confirmed that the Dunaliella salina OH214 strain of the present invention has an ability to produce neoxanthin, neoxanthin, violaxanthin, and antheraxanthin, which are xanthophyll-based pigments, chlorophyll b, chlorophyll a, and β-carotene in addition to lutein and zeaxanthin, and also has an excellent ability to produce, particularly, β-carotene.

1. Dunaliella salina OH214 (KCTC 14434BP) having an ability to produce lutein.

2. The Dunaliella salina OH214 of claim 1, wherein the Dunaliella salina OH214 is able to further produce one or more selected from the group consisting of zeaxanthin and β-carotene.

3. A culture of Dunaliella salina OH214 (KCTC 14434BP).

4. A composition comprising Dunaliella salina OH214 (KCTC 14434BP) and/or a culture thereof.

5. The composition of claim 4, wherein the composition is for oral administration, feed, feed additives, food, or food additives.

6. The composition of claim 4, wherein the composition is for enhancing or maintaining eye health; preventing or ameliorating macular degeneration; preventing or ameliorating a decline in eye function; ameliorating or preventing retinal damage; maintaining retinal health; reducing the risk of developing macular degeneration; or preventing or ameliorating vision loss.

7. A method for producing a pigment, the method comprising culturing Dunaliella salina OH214 (KCTC 14434BP).

8. The method of claim 7, wherein the method comprises extracting a pigment from Dunaliella salina OH214 (KCTC 14434BP) or a culture thereof.

9. The method of claim 7, wherein the pigment comprises one or more selected from the group consisting of lutein, zeaxanthin and β-carotene.

10. A method for producing a food raw material, the method comprising culturing Dunaliella salina OH214 (KCTC 14434BP).

11. A method for producing a feed raw material, the method comprising culturing Dunaliella salina OH214 (KCTC 14434BP).
